(19) United States
(12) Patent Application Publication
(10) Pub. No.: US 2009/0204184 A1
(43) Pub. Date: Aug. 13, 2009

(54) Light enhanced acupuncture

(76) Inventor: Frederick J. Pingal, Tucson, AZ (US)

Correspondence Address: Mark E. Ogram, Ste. 110-186, 6990 E. 22nd Street, Tucson, AZ, 85710-5192, US

(21) Appl. No.: 12/069,690

(22) Filed: Feb. 12, 2008

(51) Int. Cl. A61N 5/06

(52) U.S. Cl. 607/88

(57) ABSTRACT

An acupuncture system which gathers light from the night's sky and focuses the gathered light onto an acupuncture meridian of a patient to obtain acupuncture affects. In one embodiment of the invention, the focused light is communicated through a fiber optic cable to a hand-held wand which emits the light onto the proper acupuncture site; in another embodiment, a light needle is used to emit the light into the patient at the acupuncture site.

### BACKGROUND OF THE INVENTION

This invention relates generally to medical treatments and more particularly to acupuncture techniques used for the treatment of medical conditions.

The origins of acupuncture are shrouded in history. While the popular belief is that acupuncture originated in Asia, there is also evidence that it was practiced in Europe as early as 5000 B.C. Even though the techniques and theories have been practiced for such a long period, only recently has Western medical studies taken the practice seriously.

A meridian within the field of acupuncture, is a line connecting different anatomical sites and is used as routes for the acupuncture application.

Studies have been performed which show that the traditional sites for acupuncture (often called “meridians”), do relieve pain, while other sites do not. Also studies have shown that these same sites absorb near infra-red laser light (3 mW, 980 nm) at these acupuncture sites greater than on non-acupuncture sites.

The recognition that the acupuncture sites have some special properties have resulted in the further development of differing techniques for acupuncture. These new techniques include the application of light onto the patient to achieve therapeutic results. The use of low-level laser acupuncture has been proposed and has been implemented by several different sources.

Often, the acupuncture site is excited using electrical stimulation. In this context, a variety of different approaches have been taken, including: U.S. Pat. No. 7,155,287, entitled Device for Percutaneous Nerve Stimulation” issued to Gavronsky on Dec. 26, 2006; U.S. Pat. No. 7,200,444, entitled “Method and Device for Electro-Acupuncture” issued to Gabronsky on Apr. 3, 2007; and U.S. Pat. No. 7,308,318, entitled “Percutaneous Apparatus with Electrical Coupling” issued to Miazga et al. On Dec. 11, 2007; all of which are incorporated hereinto by reference.

Magnetic fields are also used on the acupuncture sites. One such approach is described in U.S. Pat. No. 7,167,752, entitled “Electronic Electrical and Electro-Magnetic Health Enhancement and Stimulation Device” issued to Lin-Hendel on Jan. 23, 2007, incorporated hereinto by reference.

Even lunar light has been used to bathe a patient in an effort to gain therapeutic results. Such as device is described by Chapin in the United States Patent application number US 2007/0008616 A1, entitled “Interstellar Light Collector” filed on Jan. 11, 2007, incorporated hereinto by reference.

The therapeutic affect that acupuncture achieves is not disputed.

It is clear there is a continuing need for mechanisms which enhance the body's natural healing potentials.

### SUMMARY OF THE INVENTION

This invention creates an acupuncture system which gathers light from the night's sky and focuses the gathered light onto a meridian or acupuncture point of the patient. In using the light from the night's sky, a variety of different and unique wave length combinations are available which have not been “polluted” by earth generated light nor for earth's own sun.

In the preferred embodiment, a selected star or star cluster is used from which its light is gathered. A variety of techniques are available for the gathering of the light including, but not limited to use of a telescope. In this regards, the telescope is an optical instrument which increases the angle under which a distant object is seen. Ideally, a Newtonian telescope is used. Using a reflecting concave mirror the light from the night's sky is magnified and focused into a narrow beam.

Other light collecting mechanisms are well known to those of ordinary skill in the art and include, but are not limited to, United States Patent application number US 2007/0008616 A1, entitled “Interstellar Light Collector” filed on Jan. 11, 2007, and issued to Chapin.

The collected, and focused light from the night's sky is then directed against an acupuncture site on a patient to affect acupuncture effects. Since the light is derived from distant celestial body (star(s) or planet(s)), the complexity of the wavelengths being applied is based upon the celestial characteristics.

A variety of mechanisms are well known to those of ordinary skill in the art which permit light to be focused onto a selected site on the patient. This includes, but is not limited to, U.S. Pat. No. 7,311,722, entitled “Photodynamic Stimulation Device and Methods” issued to Larsen on Dec. 25, 2007; United Kingdom Patent Application GB 2,407,174, entitled “Rainbow Projector” filed by Harries et al. on Oct. 18, 2003; and incorporated hereinto by reference.

While in some embodiments of the invention, the patient is moved to align the focused light onto the selected acupuncture site, in other embodiments, flat mirrors are used to redirect the focused light onto the selected location on the patient. This embodiment eliminates the need to move the patient and is therefore much less stressful on the patient and is also easier to apply.

In yet another embodiment of the invention, the focused light is communicated through a fiber optic cable to a hand-held wand. The operator, using the hand-held wand, is able to have the light emitted with precision onto the proper acupuncture site. In this embodiment, the requirement of moving the patient is eliminated.

In a further embodiment, a light needle is used to emit the light subcutaneously into the patient at the acupuncture site. This technique allows the selected light to be emitted within the patient, not solely cutaneously.

A variety of light needles are known to those of ordinary skill in the art. Such light needles are described in U.S. Pat. No. 5,250,068, entitled “Optical Transmission Type Acupuncture Needle” issued to Ideguchi et al. on Oct. 5, 1993; and “‘Needles of Light’: a New Therapeutic Approach” (Michael Weber, Thomas Fussganger-May, Tillman Wolf, Medical Acupuncture, 2007, 19(3) 141-150. Doi:10.1089/acu.2007.0539), both of which are incorporated hereinto by reference.

By emitting the light subcutaneously, an enhanced therapeutic affect is obtained.

The invention, together with various embodiments thereof will be more fully described by the following descriptions of the accompanying drawings.

### DRAWINGS IN BRIEF

FIG. 1 graphically illustrates an embodiment of the invention which uses a movable treatment table.

FIG. 2 is an embodiment of the invention in which the treatment table and the focusing mechanism are not in a fixed relationship.

FIG. 3 illustrates an embodiment of the invention in which a fiber-optic cable is used to communicate the patient via a handheld wand.

FIG. 4 illustrates yet another embodiment of the invention in which a light emitting needle is used with a fiberoptic communication channel.

### DRAWINGS IN DETAIL

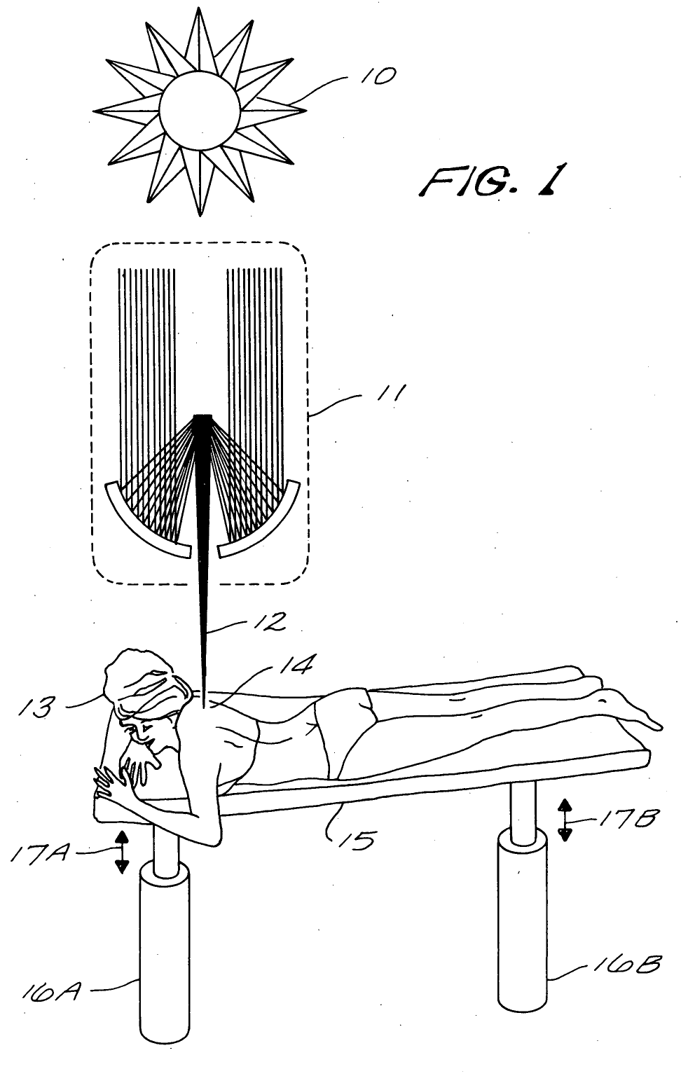

FIG. 1 graphically illustrates an embodiment of the invention which uses a movable treatment table.

In the night sky, star or planet 10 emits light rays which are focused using a Newtonian telescope 11 into beam 12. Beam 12 is directed onto a selected acupuncture location 14 on patient 13.

The light sought from the night's sky is chosen to have the desired photon characteristics so that the photon structure of the light has an emission band which affects the albumin of the blood. In this way, unique therapeutic affects are obtained. These relationships are described by Dr. Sergei Pankratov, in his article “Meridians Conduct Light” published by Raum and Zeit, in 1991, incorporated hereinto by reference.

As example, the light from star or planet 10 contains wavelengths indicative of being emanated from a source rich in iron. “Iron” wavelengths have beneficial benefits as the iron wavelength light interacts well with the iron rich hemoglobin of the patient.

Patient 13 is supported on table 15. In order to direct beam 12 to “shine” onto the appropriate acupuncture location 14, table 15 is adjusted by an operator using rams 16A and 16B which selectively move as indicated by arrows 17A and 17B. A restraining mechanism, not shown, maintains patient 13 firmly on table 15 during the movement of table 15 and also during treatment.

Those of ordinary skill in the art readily recognize a variety of other mechanisms which are capable of moving the patient so that beam 12 is directed against the proper location.

In this manner, the light is collected and directed onto a selected acupuncture location on the patient.

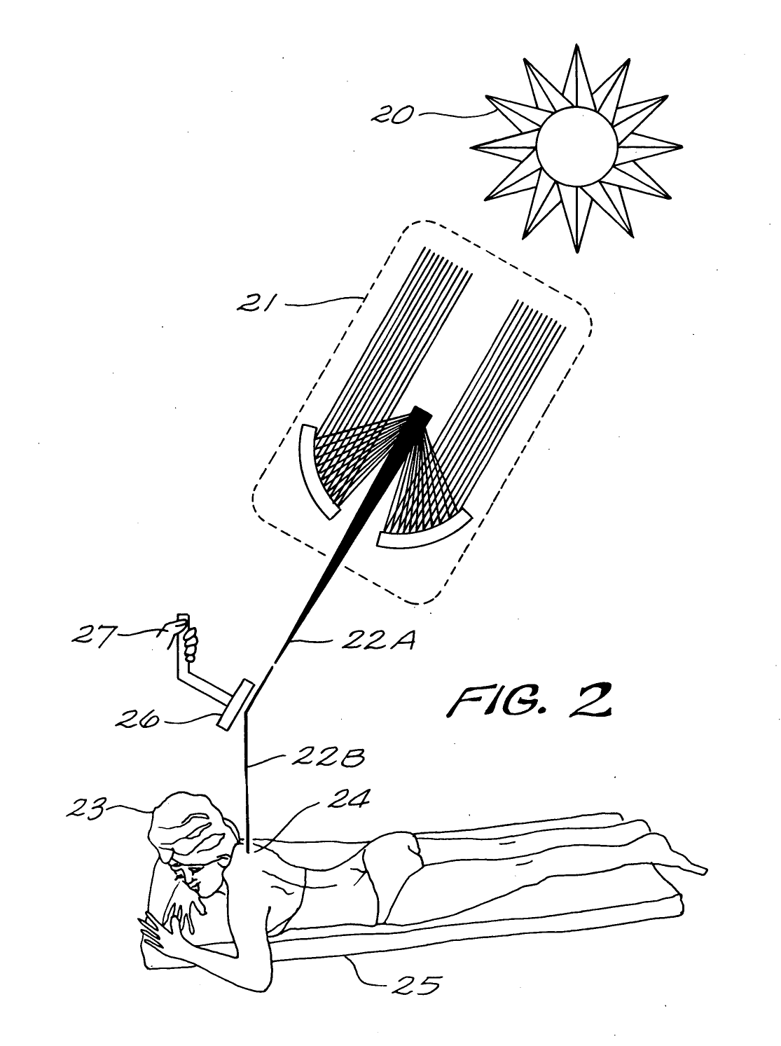

FIG. 2 is an embodiment of the invention in which the treatment table and the focusing mechanism are not in a fixed relationship.

Celestial body 20 emits light waves which are collected by focusing mechanism 21 to generate beam 22A.

Therapist 27 manually adjusts mirror 26 to redirect beam 22A so that the redirected beam 22B impinges onto a selected location 24 on patient 23. Patient 23 rests on table 25 without restraints.

In some embodiments, multiple mirrors are used to redirect the mirror or to further focus the light beam.

Through the use of mirror 26, (or multiple mirror systems), the patient does not have to be physically moved in order to achieve the proper placement of the light beam onto the patient.

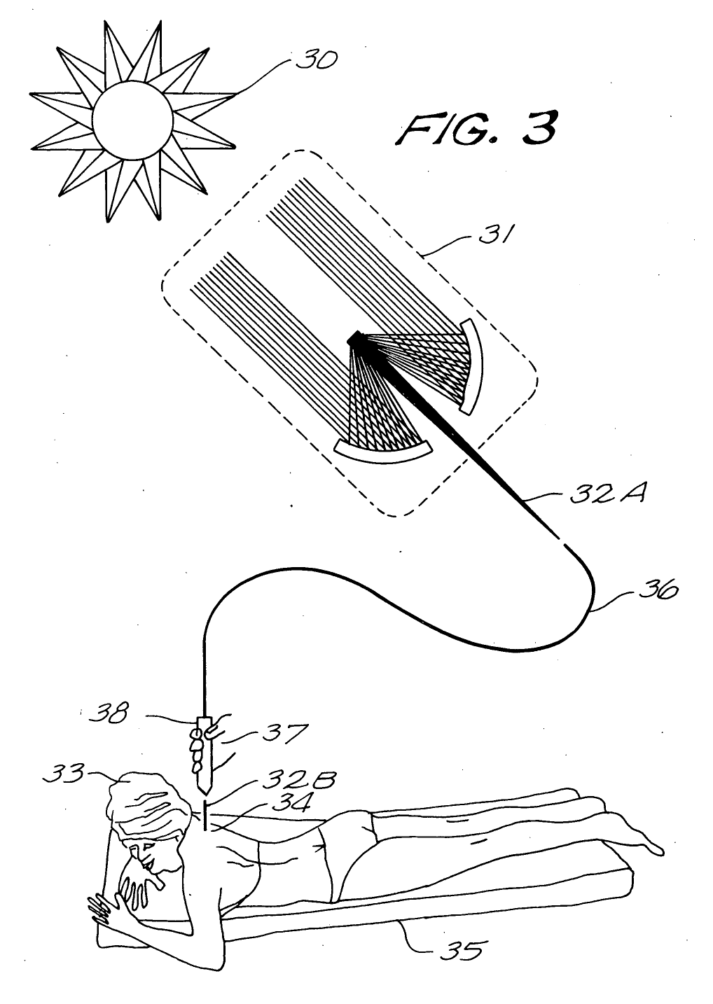

FIG. 3 illustrates an embodiment of the invention in which a fiber-optic cable is used to communicate the patient via a handheld wand.

Star or planet 30 emits light which is communicated to the Newtonian Telescope 31 which focuses the light into beam 32A. Beam 32A is communicated to fiberoptic cable 36. The light is communicated through fiberoptic cable 36, acting as a light channel, to wand 38 which is manipulated by therapist 37.

Light beam 32B is emitted from the end of wand 38 and is directed to the selected site 34 of patient 33 by therapist 37. In this way, therapist 37 is able to easily move the beam from one selected site to another for treatment.

Patient 33 rests on table 35.

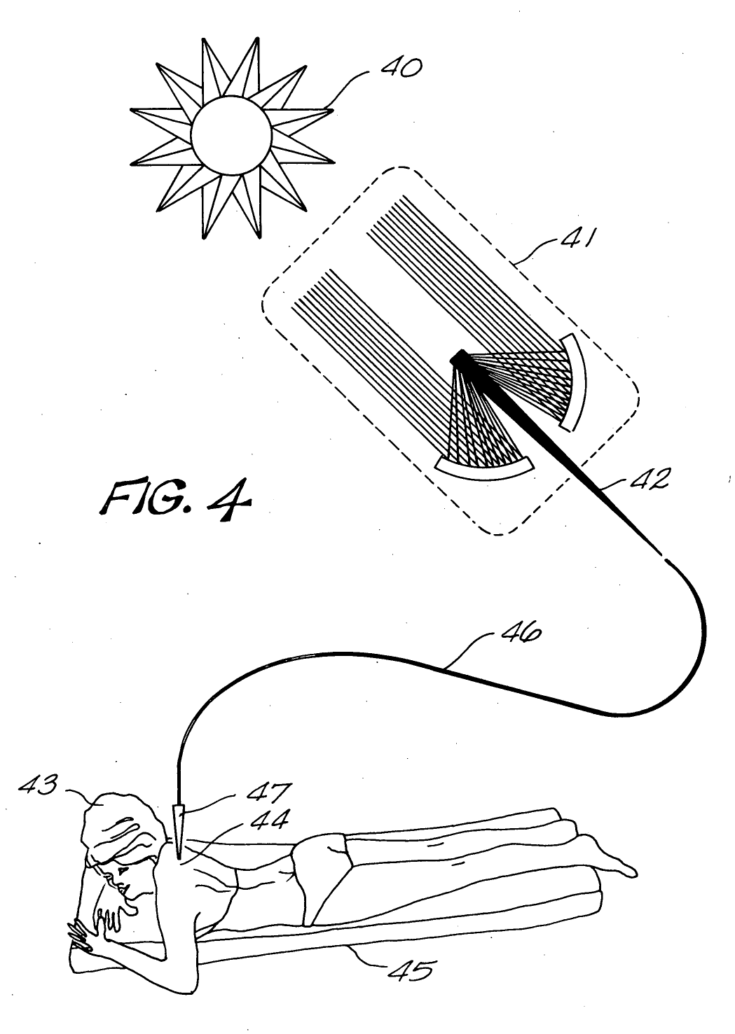

FIG. 4 illustrates yet another embodiment of the invention in which a light emitting needle is used with a fiberoptic communication channel.

Newtonian telescope 41 collects and focuses the light from the star or planet 40. This focused light beam 42 is communicated into the light channel created by fiber optic 46.

The light from fiber optic 46 is communicated to the light transmitting needle 47 which has been placed into the acupuncture site 44. This light transmitting needle 47 is ideally one as described by U.S. Pat. No. 5,250,068, described above.

Patient 43, during the treatment, rests on table 45.

It is clear that the present invention provides a mechanism to enhance the body's natural healing potentials.

What is claimed is:

1. An acupuncture system comprising a light focusing mechanism concentrating light from a selected area of a selected area of the night's sky and communicating such concentrated light onto a selected acupuncture location of a patient.

2. The acupuncture system according to claim 1, wherein the selected area of the night's sky contains a user selected celestial body.

3. The acupuncture system according to claim 2, wherein the celestial body is a planet.

4. The acupuncture system according to claim 2, wherein the celestial body is a star.

5. The acupuncture system according to claim 4, wherein the concentrated light has a wavelength reactive with albumin of blood.

6. The acupuncture system according to claim 1, wherein the selected acupuncture location lies on meridian of the patient.

7. The acupuncture system according to claim 6, further including a light pathway having a first end receiving light from said light focusing mechanism and a second end being manually manipulated to emit light onto the selected acupuncture location of the patient.

8. The acupuncture system according to claim 7, wherein the light pathway includes a fiber optic cable.

9. The acupuncture system according to claim 7,
a) further including an optical transmitting acupuncture needle; and,
b) wherein the second end of said light pathway communicates light to the optical transmitting acupuncture needle.

10. The acupuncture system according to claim 9, wherein the optical transmitting acupuncture needle is positioned within the acupuncture location.

11. An acupuncture system comprising
a) a light focusing mechanism concentrating light from a celestial body;
b) a mechanism for holding a patient; and,
c) a focusing mechanism communicating such concentrated light onto a selected acupuncture location of the patient.

12. The acupuncture system according to claim 11, wherein the focusing mechanism includes at least one manually manipulatable mirror adapted to redirect light from the light focusing mechanism to the selected acupuncture location of the patient.

13. The acupuncture system according to claim 11, wherein the celestial body is a star generating a light wavelength reactive with albumin of blood.

14. The acupuncture system according to claim 11, further including:
a) a light pathway having a first end receiving light from said focusing mechanism and a second end being manually manipulated to emit light; and,
b) an optical transmitting acupuncture needle inserted into the selected acupuncture location; and,
wherein the second end of said light pathway communicates light to the optical transmitting acupuncture needle.

15. The acupuncture system according to claim 14, wherein the light pathway includes a fiber optic cable.

16. An acupuncture system comprising:
a) a light gathering apparatus collecting light from an area of the night's sky;
b) a light focusing apparatus concentrating such collected light from the light gathering apparatus;
c) a transmission pathway communicating such focused light from the light focusing apparatus to a selected location of a patient.

17. The acupuncture system according to claim 16, wherein the selected location lies on meridian of the patient.

18. The acupuncture system according to claim 16, wherein the transmission pathway includes a fiber optic cable.

19. The acupuncture system according to claim 18, wherein an emitting end of the fiber optic cable includes a manually manipulated wand having its emitting end pressed against the skin at the selected location of a patient.

20. The acupuncture system according to claim 18,
a) further including an optical transmitting acupuncture needle; and,
b) wherein the emitting end of the fiber optic cable communicates light to the optical transmitting acupuncture needle.
